(19) United States
(12) Patent Application Publication
(10) Pub. No.: US 2005/0004664 A1
(43) Pub. Date: Jan. 6, 2005

(54) Vascular graft

(76) Inventors: Paul Martakos, Pelham, NH (US); Joseph Ferraro, Londonderry, NH (US); Theodore Karwoski, Hudson, NH (US); Ibrahim Dagher, Methuen, MA (US)

Correspondence Address: LAHIVE & COCKFIELD, LLP., 28 STATE STREET, BOSTON, MA, 02109, US

(21) Appl. No.: 10/833,930

(22) Filed: Apr. 27, 2004

Related U.S. Application Data
(60) Provisional application No. 60/466,386, filed on Apr. 29, 2003.

(51) Int. Cl. A61F 2/06

(52) U.S. Cl. 623/1.49

(57) ABSTRACT

Disclosed are vascular grafts including an expanded copolymer of tetrafluoroethylene (TFE) and perfluoropropylene vinyl ether (PPVE). In certain embodiments, the copolymer includes between about 0.01% and about 1.5% PPVE. Vascular grafts exhibit superior performance properties, e.g., radial strength, and suture strength, and manufacturing properties, e.g., sinter time. Methods of forming vascular grafts from the copolymer also are described.

### RELATED APPLICATIONS

This application claims priority to U.S. Provisional Application Ser. No. 60/466,386; filed Apr. 29, 2003.

### TECHNICAL FIELD

The present invention generally is directed to novel expanded copolymer vascular grafts. Specifically, the copolymer is an expanded copolymer of tetrafluoroethylene (TFE) and perfluoropropylene vinyl ether (PPVE).

### BACKGROUND OF THE INVENTION

Products constructed from expanded polytetrafluoroethylene (PTFE) have been disclosed, e.g., in U.S. Pat. No. 3,953,566 (Gore) and U.S. Pat. No. 4,187,390 (Gore). In these patents, it is disclosed that, while not preferred, products can also be formed from copolymers of TFE with less than 0.2% ethylene, chlorotrifluroethylene (CTFE), or hexafluoropropylene (HFP) comonomer.

Vascular grafts constructed from collagen, and/or numerous synthetic polymeric materials have been disclosed, e.g., in U.S. Pat. No. 6,520,986 (Martin et al.). Among polymers that can be used, expanded PTFE homopolymer is especially preferred, but copolymers of TFE with ethylene, PPVE, or CTFE also are mentioned.

PTFE is a homopolymer of TFE. It is a uniform, unbranched polymer having a carbon backbone and two fluorine groups bonded to each carbon. The carbon-fluorine bond is extraordinarily strong, and the homopolymer is highly crystalline due to its purity, rigidity and linearity. PTFE has a high purity because is obtained from TFE monomer by free radical polymerization without the need for additives, plasticizers, extenders and stabilizers. PTFE homopolymer typically is used in applications where its purity, corrosion resistance, insolubility and its chemical inert nature is desired or necessary (e.g., semiconductors and reactor linings). As discussed in Japanese Patent No. 42-13,560 (Shinsaburo Oshige), while PTFE has in the past been classified as a thermoplastic polymer, above its transition temperature (about 327° C.), it becomes a non-crystalline gel that cannot be adapted to conventional thermoplastic processes (e.g., extrusion, injection and pressure molding).

Accordingly, one of the disadvantages of PTFE homopolymer is that it must be processed with special equipment and techniques, because the molecular weight and melting viscosity of the homopolymer is so high. This is due, in part, to the highly crystalline nature of PTFE homopolymer. Although it is widely used, PTFE homopolymer is difficult to process and its non-adhesive nature limiting. Various methods of making PTFE articles are known and have been described, e.g., in U.S. Pat. No. 5,433,909 (Martakos et al.) and U.S. Pat. No. 5,474,824 (Martakos et al.).

Non-expanded copolymer TFE-PPVE copolymer currently is employed in industrial or electrical applications as a heavy-duty corrosion-resistant protective material (e.g., polymer linings for conduits and tanks in chemical plants, and wire and cable coatings). It also has been used to protect instruments (e.g., temperature probes) in high-temperature, corrosive environments (e.g., semi-conductor applications).

### BRIEF SUMMARY OF THE INVENTION

The present invention is directed to medical devices, e.g., vascular grafts, formed from expanded TFE-PPVE copolymers. Vascular grafts constructed from these copolymers have surprisingly exhibited marked and distinct improvements in performance and manufacturing characteristics. For example, the copolymer grafts are less susceptible to radial stress (which can lead to rupture or tears), and stress caused by suture tension (which can lead to misplacement of grafts), as compared to conventional grafts. Such failures can lead to additional surgeries to replace the compromised graft, thereby increasing risk to the patient and prolonging healing time. The copolymer grafts also are advantageous from a manufacturing perspective, as they are easier to process. For example, the sintering time can be significantly reduced as demonstrated in the example below. It is believed that copolymer grafts will further provide significant healing benefits.

In one aspect, the present invention generally is directed to a vascular graft comprising an expanded copolymer comprising polymerized TFE monomer units and PPVE monomer units. The copolymer can include between about 0.01% and about 1.5%, between about 0.05% and about 0.5%, or about 0.1% PPVE.

In certain embodiments, the vascular graft has at least a 1.5-fold, 1.75-fold, or a 2-fold increase in Radial Burst Test (RBT) pressure versus a comparable PTFE homopolymer graft. In certain embodiments, the vascular graft has at least a 2-fold, 3-fold, or 4-fold increase in Suture Retention Test (SRT) strength versus a comparable PTFE homopolymer graft. In certain embodiments, the vascular graft has at least a 1.5-fold increase in Radial Tension Strength (RTS) versus a comparable PTFE homopolymer graft. The vascular grafts of the invention can have low Water Entry Pressure (WEP), and/or a transition point between about 324° C. and 325° C.

In another aspect, the invention is directed to a vascular graft comprising an expanded copolymer including polymerized TFE and PPVE monomer units. The vascular graft has at least a 2-fold increase in RBT and/or a 4-fold increase in SRT, and/or a 1.5-fold increase in RTS versus a comparable PTFE homopolymer graft.

In another aspect the vascular graft consists essentially of an expanded copolymer of polymerized TFE monomer units and PPVE monomer units. The vascular graft copolymer can include between about 0.01% and about 1.5% PPVE.

In yet another aspect, the invention is generally directed to a method of forming a vascular graft. The method includes the step of forming a vascular graft from a copolymer resin comprising polymerized TFE monomer units and PPVE monomer units. The copolymer can include between about 0.05% and about 0.5% of the copolymer, or about 0.1% of the copolymer. The graft can consist essentially of or entirely of the copolymer.

Finally, the apparatus and methods of the invention are not limited to vascular grafts. The copolymers of the present invention can also be employed to form a wide variety of devices including, but not limited to, medical, veterinary, and dental devices as disclosed in greater detail below.

Additionally or alternatively, the method, graft and copolymer can have any of the attributes described or claimed herein.

### BRIEF DESCRIPTION OF THE DRAWINGS

The subject matter regarded as the invention is particularly pointed out and distinctly claimed in the concluding portion of the specification. The invention, however, both as to organization and method of practice, together with further objects and advantages thereof, is best understood by reference to the following illustrative descriptions taken in conjunction with the accompanying Figures.

FIGS. 1A-C are schematic representations of three exemplary vascular graft configurations that can be formed in accordance with the teachings of the invention.

FIG. 2 is a graph comparing the heat flow characteristics of TFE-PPVE copolymer grafts (solid line) and PTFE homopolymer graft (dashed line).

FIG. 3 is a graph comparing the heat flow characteristics of TFE-PPVE copolymer resin (dashed line) and PTFE homopolymer resin (solid line).

### DETAILED DESCRIPTION OF THE INVENTION

The present invention provides novel vascular grafts constructed, at least in part, from an expanded copolymer of TFE and PPVE. The grafts of the invention exhibit unexpectedly improved properties over conventional grafts formed from expanded PTFE homopolymer. The vascular grafts of the invention exhibit markedly superior radial strength, thus allowing it to withstand internal and external radial expansion and compression stresses that conventional grafts cannot. The vascular grafts of the invention also exhibit improved kink resistance, further enhancing the ability of the graft to remain patent as reduction in flow cross-sectional area can adversely affects patency. Suture strength is also greatly improved in the grafts of the present invention, as well as the ability to adhere the graft to complementary members or devices.

The copolymers of the present invention can also be employed to form a wide variety of devices including, but not limited to, medical, veterinary, and dental devices. Such devices can take any form (e.g., tapes, mesh, films, laminates, tubes, and rods). The copolymer can be used to form, at least in part, non-vascular grafts, surgical mesh, stents (e.g., vascular, and coronary stents), drains (e.g., chest drains), incontinence devices, retention products, implants (e.g., facial implants), shunts (e.g., ocular shunts), and catheters (e.g., thoracic, angioplasty, cardiovascular, neurovascular, gastroenterological, nephrological, peripheral, neurological, and venous catheters). The copolymer of the present invention can also be used as a coating any of the above devices. Such a coating can be applied to provide various advantageous properties, including but not limited to, providing a lubricous surface, biocompatibility, versatility, and protection from electronic interference.

As used herein, the term “copolymer” refers to a polymer formed by polymerization of at least two monomers. The percentage of co-monomer in a copolymer, as used herein, refers to the percentage of PPVE co-monomer units incorporated into the copolymer. That is, if there is an average of about 1 PPVE mer every 1000 mers in the copolymer (including both TFE and PPVE units), there is about 0.1% PPVE in the copolymer. The term “homopolymer” refers to a polymer formed from only one monomer.

As used herein, “a comparable PTFE homopolymer graft” is used in the context of comparing the properties of the TFE-PPVE copolymer grafts of the invention to conventional PTFE homopolymer grafts. In the context of the Radial Burst Test, the Suture Retention Test, Radial Tension Strength, and water entry pressure, a comparable PTFE homopolymer graft is a graft having a comparable wall thickness and porosity to that of the copolymer graft.

The following mechanical measurements were used to characterize the vascular grafts of the present invention and compare them to vascular grafts constructed from PTFE homopolymer.

Water Entry Pressure (WEP) is defined as the pressure value necessary to push water into the pores of a synthetic tubular substrate and can be classified as High (>400 mm Hg), Medium (200-400 mm Hg), or Low (<200 mm Hg). To compute WEP, the material is subjected to an incrementally increasing water pressure until small beads of water appear on the surface.

Longitudinal Tensile Strength (LTS) is a measure of the force required to stretch or extend a graft in a radial direction.

Radial Tensile Strength (RTS) is a measure of the force required to stretch or extend a graft in a radial direction. The test mimics a pressure load on the graft similar to taking a rubber band and stretching it between a finger of each hand from the inside of the graft. For grafts, RTS can be more important than LTS as a graft typically is more likely to undergo radial stress than longitudinal stress.

Suture Retention Test (SRT) is a measure of the force required to break or rip a suture out from the end of a graft at its anastomosis. The test uses a 1 mm bit from the end of the graft and a 5.0 suture. Force is applied to the suture in a longitudinal direction from the end of the suture.

The Radial Burst Test (RBT) is a measure of the force required to burst the graft.

The “Inside Internodal Distance” is a measure of the internal porosity (at the flow surface), and the “Outside Internodal Distance” is a measure of the external porosity (at the vascular wall surface). Certain expanded polymeric materials, including PTFE and the TFE-PPVE copolymers of the present invention are characterized by lengthwise-oriented fibrils interrupted by transverse nodes. The pore size in microns is typically determined by measuring fiber length between the nodes (internodal distance). To compute fibril length, the material is viewed under sufficient magnification. A fibril length is measured from one edge of one node to the edge of an adjacent node. Fibril lengths are measured from the sample to compute a mean fibril length.

Nodes and fibrils may be further characterized by their relative geometry. That is, nodes by length, width, and height; and fibrils, by diameter and length. It is the relative geometry of nodes to fibrils, as well as, internodal distance and fibril density that determines porosity and permeability of the porous structure. The physical space between connecting nodes is composed of solid thread like fibers called fibrils in conjunction with a gaseous void volume. Fibril density refers to the relative volume consumed by fibrils between the nodes. The internodal distances and wall thickness greatly impact the performance of a graft, e.g., the feel, suturability, and healing ability of the graft.

The above described test methods for WEP, LTS, RTS, SRT, RBT and internodal distances are known to skilled artisans. All of these tests can be normalized to wall thickness and cross-sectional area. Moreover, detailed guidelines and standards for these measurements are available from the American Society for Testing Materials (ASTM) and the Association for the Advancement of Medical Instrumentation (AAMI).

The enthalpy and the transition temperature(s) of polymers can be determined in accordance with several test methods, including Differential Scanning Calorimetry (DSC). DSC is a technique that determines the variation in the heat flow into or out of a sample as it undergoes temperature scanning in a controlled atmosphere. DSC also allows the determination of thermal transition points.

The novel vascular grafts of the present invention are constructed from expanded TFE-PPVE copolymers. Vascular grafts constructed from these copolymers have surprisingly exhibited marked and distinct improvements in performance characteristics, such as radial burst strength, and suture retention strength.

The PPVE co-monomer in the TFE-PPVE copolymers of the present invention can be present in an amount between about 0.003% and 4% of the copolymer. All values and ranges included or intermediate within the ranges set forth herein also are intended to be within the scope of the present invention. For example, copolymer can include btween about 0.01% and about 1.5%, between about 0.01% and about 1%, between about 0.05 and 0.5%, and about 0.1% PPVE. In another example, the PPVE co-monomer can be present in an amount of about 0.01%, 0.02%, 0.03%, 0.04%, 0.05%, 0.06%, 0.07%, 0.08%, 0.09%, 0.1%, 0.11% . . . 1.5% in the copolymer.

Suitable non-expanded copolymer resin that can be expanded and used to construct the vascular grafts of the present invention is available from Daikin (Orangeburg, N.Y.) under the trade designation F-301. Other suitable copolymers include those available from E.I. du Pont de Nemours and Company (Wilmington, Del.) under the trade designation Teflon NXT. These copolymers allow a thinner wall diameter, improved weldability, and improved resistance to deformation under load. Also suitable are copolymers available from 3M (St. Paul, Minn.) under the trade designation Dyneon TFM PTFE, and Asahi Glass Company (Tokyo, Japan) under the trade designation CD-086.

There are several significant differences in the properties of the TFE-PPVE copolymer that demonstrate its superiority as compared to PTFE homopolymer. For example, as demonstrated in Table 3 below, grafts made from TFE-PPVE copolymer exhibit a significant improvement in Radial Tensile Strength, an over 4-fold improvement in Suture Retention Test strength, and a 2-fold improvement in Radial Burst Test strength versus PTFE grafts. Moreover, the TFE-PPVE copolymer grafts are more kink resistant than PTFE grafts. Radial strength, stitch retention, and kink resistance are common causes of failure in vascular grafts. Accordingly, the novel vascular grafts of the present invention represent a significant improvement in the art.

The vascular grafts of the invention can exhibit at least a 1.5-fold, 1.75-fold, 2-fold, and/or a 3-fold increase in RBT versus a comparable PTFE homopolymer graft. All values and ranges included or intermediate within the values and ranges set forth herein are also intended to be within the scope of the present invention. For example the RBT can be at least 1.6, 1.7, 1.8 . . . 2.5-fold that of a comparable PTFE homopolymer graft.

The vascular grafts of the invention can exhibit at least a 2-fold, 3-fold, 4-fold, and/or a 5-fold increase in SRT versus a comparable PTFE homopolymer graft. All values and ranges included or intermediate within the values and ranges set forth herein are also intended to be within the scope of the present invention. For example the SRT can be at least 2.1, 2.2, 2.3 . . . 4.5-fold that of a comparable PTFE homopolymer graft.

The vascular grafts of the invention can exhibit at least a 1.5-fold, 1.75-fold, 2-fold, and/or a 3-fold increase in RTS versus a comparable PTFE homopolymer graft. All values and ranges included or intermediate within the values and ranges set forth herein are also intended to be within the scope of the present invention. For example the RTS can be at least 1.6, 1.7, 1.8 . . . 2.5-fold that of a comparable PTFE homopolymer graft.

Additionally or alternatively, the vascular grafts of the invention can feature a WEP of less than 250 mm Hg, for example, less than 250, 225, 200, or 190 mm Hg. All values and ranges included or intermediate within the values and ranges set forth herein are also intended to be within the scope of the present invention. The WEP can be a low WEP or a medium WEP. In the latter case, preferably the WEP is on the lower end of the range for medium WEP.

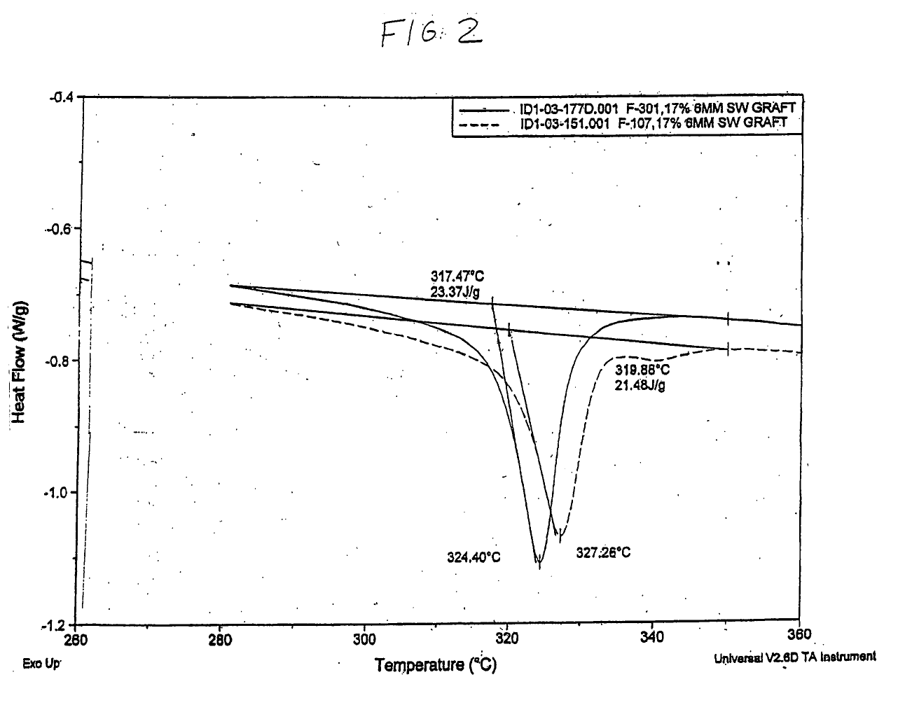

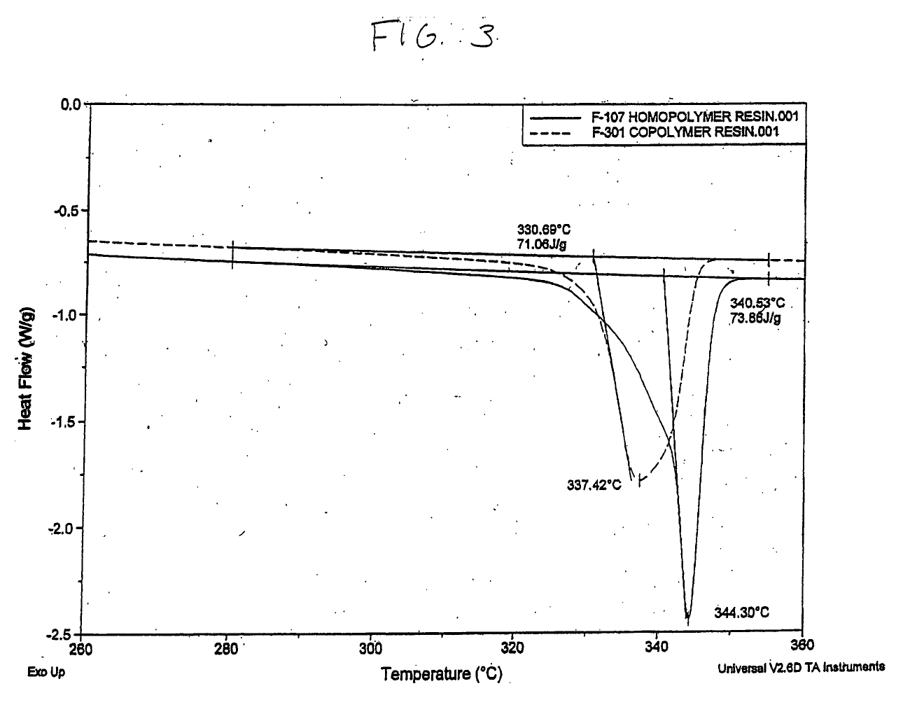

FIG. 2 and FIG. 3 are graphs generated using DSC techniques to analyze grafts and resin, respectively for both PTFE homopolymer and TFE-PPVE copolymer. As FIG. 2 demonstrates, vascular grafts formed from expanded copolymer having approximately 0.1% PPVE have a thermal transition point less than that for conventional expanded PTFE homopolymer. Specifically, the copolymer graft had a transition point of approximately 324.4° C., and the homopolymer graft had a transition point of approximately 327.3° C.

Copolymer vascular grafts of the present invention have a transition temperature lower than that of homopolymer PTFE grafts. For example, the copolymer grafts of the present invention can have a transition point of less than about 326° C., 325° C., 324° C., or 323° C. All values and ranges included or intermediate within the values and ranges set forth herein are also intended to be within the scope of the present invention. For example, the transition point can be between about 324.0° C. and 325.0° C., or about 324.5° C.

The articles of the present invention can be formed in whole or in part from the copolymer of the present invention. That is, the TFE-PPVE copolymer can be used to form the entire article (e.g., an entire graft), a portion of the article, essentially the entire article, or the entire articles.

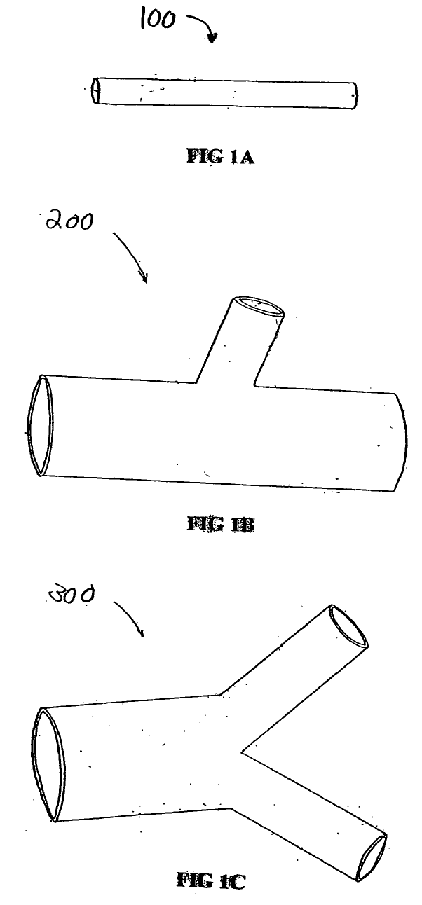

A method of making the vascular grafts of the invention will now be described with reference to exemplary embodiments. Cylinders, tubes, sheets, tapes, webs, meshes, and other shapes can be created by either of these embodiments to form all or part of a vascular graft. FIGS. 1A, 1B, and 1C, depict exemplary configurations of vascular stents 100, 200, and 300, respectively. The configurations depicted in FIGS. 1A-C are presented to illustrate three of many possible configurations that are within the scope of the invention.

The embodiments involve the use of expandable polymers. Although expandable polymer material may be prepared in a variety of ways, one method involves the use of wettable liquid or lubricant, to aid an initial extrusion process. A wettable liquid is capable of entering the pores of the expandable polymer resin. The invention is not limited to expandable polymers prepared by extrusion, or by the use of a wettable liquid for extrusion.

By way of example, an expandable polymer resin, including any of the TFE-PPVE resins disclosed herein, may be blended with a lubricant. The lubricant can be any of various polymer processing lubricants known to skilled practitioners, including aliphatic hydrocarbon extrusion aids such as odorless mineral spirits. One suitable lubricant is ISOPAR H mineral spirits from Exxon Chemical Company (Houston, Tex.).

Mixtures of lubricants or wetting agents can also be used. For example, a mixture of ISOPAR mineral spirits and polyethylene glycol can be utilized. Polyethylene glycol may be preferred for certain in vivo applications because it is a biocompatible liquid. Naphtha, poly lactic acid, and alchohol and water, are additional examples lubricants that may be used within the scope of the invention, alone or in combination with one or more additional lubricants.

The lubricant-resin mixture can further include one or more drugs or agents desired to be incorporated into the expandable polymer. The combination of the resin with the drugs and agents can occur before, during, with, or after the addition of the lubricant to the resin. Moreover, the lubricant can be chosen for compatibility (e.g., stability, solvency or miscibility), with one or more drugs or agents. For example, the combination of Heparin and polyethylene glycol can be utilized. The expanded polymer resulting from the use of this combination can release Heparin in a controlled manner. The rate of release of the drug or agent also can be varied by altering the volumes, ratios, and/or contents of the mixtures. Other drugs or drug agents can be incorporated into the lubricant for use in accordance with the teachings of the present invention. Table 1 provides exemplary drugs/agents suitable for use in accordance with the teachings of the present invention:

TABLE 1
Exemplary Drugs/Agents Suitable
For Combination With the Vascular Grafts of the Present Invention

| Class | Examples |
|---|---|
| Antioxidants | Alpha-tocopherol, lazaroid, probucol, phenolic |
| | antioxidant, resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium |
| | phosphate |
| Growth Factor Antagonists | Angiopeptin, trapidil, suramin |
| Antiplatelet Agents | Aspirin, dipyridamole, ticlopidine, clopidogrel, GP |
| | IIb/IIIa inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, |
| Metabolism (e.g. statins) | amlopidine |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, ranpirnas, |
| | troglitzon, valsarten, pemirolast |
| Tissue growth stimulants | Bone morphogeneic protein, fibroblast growth factor |
| Gasses | Nitric oxide, super oxygenated O2 |
| Promotion of hollow organ | Alcohol, surgical sealant polymers, polyvinyl particles, |
| occlusion or thrombosis | 2-octyl cyanoacrylate, hydrogels, collagen, liposomes |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | snap |
| Gases | Nitric oxide, super oxygenated O₂ compound solutions |
| Imaging Agents | Halogenated xanthenes, diatrizoate meglumine, |
| | diatrizoate sodium |
| Anesthetic Agents | Lidocaine, benzocaine |
| Descaling Agents | Nitric acid, acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for |
| | enhancing medication tissue absorption, distribution and |
| | permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |

The lubricant may be mixed with the resin to control the degree of material shear that occurs during subsequent extrusion and to prevent excessive shear, which can damage the material. By application of pressure, the lubricated powder may then be preformed into a billet.

Using a ram-type extruder, the billet may be extruded through a die having a desired cross-section, typically a circle, thereby forming a cylinder. A variety of shapes may be formed by extrusion, such as a solid or hollow cylinder, a flat sheet, a rectangle and the like. The tubing can then be cut into desired lengths and the ends secured for handling (e.g., metal plugs are inserted into the ends of the tubes and clamped or otherwise affixed to the plugs).

The lengths are then stretched. Stretching can be performed in more than one direction. Stretching is typically performed, in the case of a cylinder, by applying tensile force to the ends of the cylinder. In the case of a flat sheet, stretching is typically performed in the machine direction. Alternatively, or in addition, stretching may be performed in the radial or transverse direction to a cylinder or flat sheet, respectively. For example, in the case of a hollow cylinder, a mandrel may be used to radially stretch the hollow polymer cylinder. Tensile force may be applied to stretch the cylinder simultaneously with the use of a mandrel or at a different time. Within the scope of the invention, a combination of various stretching may be combined or applied in succession.

Additional or alternatively, by appropriately controlling the temperature and time conditions to be employed for stretching operations, along with the arrangement of zones within the wall cross-section, the graft can be provided with a profile of gradual change in its fibrous structure through the thickness of the tube wall wherein the porous structure of the inner surface is separated from the outside surface

Heat may also be applied to the expandable polymer prior to or during stretching. It may be preferable to keep the temperature of the expandable polymer below a boiling point of the lubricant to inhibit loss of the lubricant. It is further preferable to keep the temperature of the expandable polymer and the lubricant below a degradation point of any drugs or agents incorporated into the expandable polymer and lubricant.

After removal of lubricant, the extruded tube is expanded and sintered, according to the known methods including, but not limited to, the methods described in the US patents cited herein and incorporated herein by reference, under various conditions to produce material with different node/fibril structures. By way of example, the tube lengths can then secured against contraction and sintered in an oven above the copolymer transition temperature to fix the node and fibril structure induced by stretching. The tube lengths can then be cut to a desired length and sterilized for use. Various known techniques can also be used to further modify the graft (e.g., a tapered graft can be made by use of a mandrel heated to a temperature at which the graft will expand). Alternative techniques for manufacturing expanded polymer grafts can also be employed.

The embodiments and their variations described above are intended to be representative of the scope of the invention and not limiting. It is also intended to be within the scope of the invention for variations of the embodiments to be applicable to other embodiments.

The invention will now be described with respect to various examples involving various forms, beginning with sheets and films.

### EXAMPLE

Vascular grafts were made from PTFE homopolymer and a TFE-PPVE copolymer resins. The PTFE homopolymer (F-107) and PTFE-PPVE (F-301) copolymer resins were both obtained from Daikin America (Orangeburg, N.Y.). The TFE-PPVE copolymer included about 0.1% PPVE. The lubricant, ISOPAR odorless mineral spirits, was obtained from Exxon Chemical Company (Houston, Tex.).

As shown, e.g., in FIGS. 2 and 3, there are significant differences between the properties between the homopolymer and copolymer resins and expanded polymers. Homopolymer resins have a higher transition point (about 344° C.) compared to the copolymer resin (about 337° C.). Grafts made from the resins also show differences between transition points. Grafts made from homopolymer resin have a higher transition point of (about 327° C.) than grafts made from copolymer resin (about 324° C.).

A paste was formed with homopolymer and copolymer resin particles in approximately 17% lubricant by weight. The paste was extruded to form tubing. The tubing was cut into lengths and the ends were secured for handling. The secured lengths then were heated in an oven. After removal from the oven, the tube lengths were stretched. The tube lengths were then secured against contraction and sintered in an oven. The copolymer graft sintered more quickly than the homopolymer graft. Process conditions were chosen to achieve equivalent porosity (internodal distance) between samples. As demonstrated in Table 2, this required a significant difference in expansion and sinter conditions between resins in order to achieve equivalent porosity. The copolymer grafts required significantly less sintering time than the homopolymer grafts.

TABLE 2
Processing Conditions for Vascular Grafts Made From
Expanded TFE-PPVE Copolymer Versus PTFE Homopolymer

| | TFE-PPVE | |
|---|---|---|
| | Copolymer | PTFE Homopolymer |
| Initial Stretch Length (in) | 16 | 11 |
| Final Stretch Length (in) | 45 | 45 |
| Stretching Velocity (ips) | 29 | 5 |
| Stretch Temperature (° C.) | 290 | 320 |
| Stabilizing Time at 290° C. (sec) | 300 | 300 |
| Sintering Time (sec.) | 30 | 180 |
| Sintering Temperature (° C.) | 360 | 360 |

The grafts were tested for the mechanical properties listed in Table 3. As Table 3 demonstrates, the copolymer grafts are significantly different from the homopolymer graft. For example, the copolymer grafts exhibited superior strength in the radial direction as compared to the homopolymer graft: the Radial Tensile Strength (RTS) of the copolymer graft was 60% stronger than the homopolymer graft. Moreover, the burst pressure for the copolymer graft was double that of the homopolymer graft. The TFE-PPVE copolymer graft also exhibited a vastly superior suture retention strength: It was well over four times stronger than that measured for the PTFE homopolymer graft. Also, the density of the copolymer grafts was significantly higher than that of the homopolymer grafts.

TABLE 3
Mechanical Data Comparing Vascular Grafts Made From
Expanded TFE-PPVE Copolymer Versus PTFE Homopolymer

| | TFE-PPVE | PTFE |
|---|---|---|
| | Copolymer | Homopolymer |
| Density (g/cc) | 0.770 | 0.502 |
| WEP (mm Hg) | 185 | 266 |
| LTS (lbs) | 23 | 60 |
| RTS (lbs) | 48 | 30 |
| SRT (lbs) | 1.9 | 0.4 |
| RBT (psi) | 82 | 41 |
| Wall (in) | 0.025 | 0.0245 |
| ID (in) | 0.242 | 0.241 |
| Inside Internodal Distance | 30 | 27 |
| (microns) | | |
| Outside Internodal | 24 | 31 |
| Distance (microns) | | |
| DSC Enthalpy (J/g) | 23.37 | 21.43 |
| DSC Transition Temp (C) | 324.4 | 327.9 |

Based on this data, it is believed that the copolymer grafts of the invention will exhibit superior performance in use, as they are less susceptible to radial stress (which can lead to rupture or tears), and stress caused by suture tension (which can lead to misplacement of grafts). Such failures often lead to additional surgeries to replace the compromised graft leading to increased risk to the patient and prolonged healing time. The copolymer grafts also are advantageous from a manufacturing perspective, as they are easier to process. For example, the sintering time is reduced to 30 seconds, as compared to the 180 seconds required for comparable homopolymer grafts. It is believed that copolymer grafts will further provide a significant benefit in healing time.

The vascular grafts of the present invention have wide ranging applications, such as devices for in vivo implantation, prostheses intended for placement or implantation to supplement or replace a segment of a natural biological blood vessel, and supports for tissue repair, reinforcement or augmentation.

Since certain changes may be made in the above constructions and the described methods without departing from the scope of the invention, it is intended that all matter contained in the above description or shown in the accompanying drawings be interpreted as illustrative and not in a limiting sense. By way of example, any known methods for varying the porosity and/or chemistry characteristics of implantable prostheses, such as varying the lubrication level in the blended pasted, viewed in combination with the disclosed methods are considered to be within the scope of the present invention. Additionally, any methods for combining resins, pastes, billets or extrudates, according to the methods of the invention, are also considered to be within the scope of the present invention.

All patents and references identified in this application are hereby incorporated by reference herein. Having described the invention,

1. A vascular graft comprising an expanded copolymer comprising polymerized tetrafluoroethylene (TFE) monomer units and perfluoropropylene vinyl ether (PPVE) monomer units, wherein the PPVE monomer units comprise between about 0.01% and about 1.5% of the copolymer.

2. The vascular graft of claim 1, wherein the copolymer comprises between about 0.05% and about 0.5% PPVE.

3. The vascular graft of claim 1, wherein the copolymer comprises about 0.1% PPVE.

4. The vascular graft of claim 1, having at least a 1.5-fold increase in Radial Burst Test (RBT) pressure versus a comparable PTFE homopolymer graft.

5. The vascular graft of claim 1, having at least a 1.75-fold increase in RBT versus a comparable PTFE homopolymer graft.

6. The vascular graft of claim 1, having at least a 2-fold increase in RBT versus a comparable PTFE homopolymer graft.

7. The vascular graft of claim 1, having at least a 2-fold increase in Suture Retention Test (SRT) strength versus a comparable PTFE homopolymer graft.

8. The vascular graft of claim 1, having at least a 3-fold increase in SRT versus a comparable PTFE homopolymer graft.

9. The vascular graft of claim 1, having at least a 4-fold increase in SRT versus a comparable PTFE homopolymer graft.

10. The vascular graft of claim 1, having at least a 1.5-fold increase in Radial Tension Strength (RTS) versus a comparable PTFE homopolymer graft.

11. The vascular graft of claim 1, wherein the graft has a low Water Entry Pressure.

12. The vascular graft of claim 1, wherein the graft has a transition point between about 324° C. and about 325° C.

13. A vascular graft comprising an expanded copolymer comprising polymerized TFE and PPVE monomer units, the vascular graft having at least one favorable property selected from the group consisting of: at least a 2-fold increase in RBT, at least a 4-fold increase in SRT, and at least a 1.5-fold increase in RTS, versus a comparable PTFE homopolymer graft.

14. The vascular graft of claim 13, having at least two favorable properties selected from the group consisting of: at least a 2-fold increase in RBT, at least a 4-fold increase in SRT, and at least a 1.5-fold increase in RTS, versus a comparable PTFE homopolymer graft.

15. The vascular graft of claim 13, having at least a 2-fold increase in RBT, at least a 4.5-fold increase in SRT, and at least as 1.5-fold increase in RTS, versus a comparable PTFE homopolymer graft.

16. A vascular graft consisting essentially of an expanded copolymer of polymerized TFE monomer units and PPVE monomer units.

17. The vascular graft of claim 16, wherein the copolymer comprises between about 0.01% and about 1.5% PPVE.

18. A method of forming a vascular graft, the method comprising the step of forming a vascular graft from a copolymer resin comprising polymerized TFE monomer units and PPVE monomer units.

19. The method of claim 16, wherein the copolymer comprises between about 0.05% and about 0.5% PPVE.

20. The method of claim 16, wherein the copolymer comprises about 0.1% PPVE.

21. A medical device comprising an expanded copolymer comprising polymerized TFE monomer units and PPVE monomer units, wherein the PPVE monomer units comprise between about 0.01% and about 1.5% of the copolymer.
